(19) United States
(12) Patent Application Publication
(10) Pub. No.: US 2006/0258973 A1
(43) Pub. Date: Nov. 16, 2006

(54) Micro-current Iontophoretic Percutaneous Absorptive Patch

(76) Inventor: Kevin Volt, Atlantic City, NJ (US)

Correspondence Address: KEVIN VOLT, 2717 FAIRMONT AVE., ATLANTIC CITY, NJ, 08401, US

(21) Appl. No.: 10/908,107

(22) Filed: Apr. 27, 2005

(51) Int. Cl. A61B 17/20

(52) U.S. Cl. 604/22

(57) ABSTRACT

A self-contained, conveniently disposable percutaneous absorptive patch of the present invention utilizes, in a novel ways, the existing percutaneous absorptive principles to delivering topical medications quickly, painlessly, and effectively through the skin barrier. The absorptive principles include the epidermal microcuts/micro-needles, permeation enhancers, and micro-current iontophoresis. Most importantly, the transdermal absorptive patch of the present invention introduces a novel incorporation of self-producing micro-electrical currents and micro-electrodes into the existing conventional iontophoretic principle.

### STATEMENT REGARDING FEDERALLY SPONSORED RESEARCH OR DEVELOPMENT

Not Applicable.

### BACKGROUND OF THE INVENTION

1. Field of the Invention

The present invention generally concerns transdermal delivery of drugs by using a self-contained, disposable skin patch. The dermal patch of the present invention utilizes both passive and active methods for delivering therapeutic agents across the skin barrier. Passive means that the delivery system relies on natural factors such as diffusion coefficient, lipophilic solubility, and/or concentration gradients for transportation and delivery of therapeutic agents across the skin barrier. Active means utilizing energy in general, direct electric current in particular, to facilitate delivering of therapeutic agents across the skin barrier.

2. Background of the Related Arts

Structure of the Skin Barrier

The skin is the largest human organ and consists of three functional layers: epidermis, dermis, and subcutaneous. It has a wide variety of functions. One major task of the skin is to protect the organism from water loss and mechanical, chemical, microbial, and physical influences. The protective properties are provided by the outermost layer of the skin, the epidermis. Although its thickness measures on average only 0.1 mm (from 0.02 mm on the face up to 5 mm on the soles of the feet) it is specially structured to fulfill this challenging task. Out of the five layers of the epidermis, it is mainly the uppermost layer, the stratum corneum (the horny layer), which forms the permeability barrier.

The stratum corneum consists of horny skin cells (corneocytes) which are connected via desmosomes (protein-rich appendages of the cell membrane). The corneocytes are embedded in a lipid matrix. Thus the structure of the stratum corneum can be roughly described by a “brick and mortar” model. The corneocytes of hydrated keratin comprise the bricks and the epidermal lipids fill the space between the dead cells like mortar. In order for percutaneous absorption to occur the chemical must pass through this tough layer and reach the living epidermis. Once a chemical gains access to the dermis, rapid and complete absorption is usually assured.

Routes of Percutaneous Absorption

Two diffusional routes exist for a molecule to penetrate normal intact skin: across the intact epidermis or via the skin appendages (hair follicles and sweat glands). However, the appendages have a small fractional surface area (approximately 0.1%) of the total skin area and are believed to provide an insignificant pathway for most drug penetration. The majority of agents permeate thus across the epidermis, in which a molecule must initially partition into the stratum corneum before diffusing across the viable epidermis. Two micropathways may exist through the stratum corneum; the transcellular and intercellular routes. The principal pathway for a penetrant is determined by factors such as the diffusant partition coefficient. Hydrophilic molecules may preferentially partition into the intracellular domains, while lipophilic ones may traverse the corneum via the intercellular route. In fact, most molecules penetrate through the corneum by both routes.

The thickness of the skin, especially the stratum corneum, also determines the degree to which substances are absorbed. Thicker skin is a greater barrier to passage of foreign substances. Depending on skin thickness, there can also be variability in absorption of a given substance by different regions of the body. For example, hydrocortisone is absorbed over 50-times greater by genital skin versus the skin of the palms.

Therefore, altering the integrity of the stratum corneum, both through artificial interference or direct environmental influence, can also alter the barrier properties of skin and enhance absorption of substances. Even something as innocuous as the removal of outer layers of skin with cellophane tape can apparently dramatically increase dermal absorption.

Penetration Enhancement

The perfect barrier properties of the epidermis restricts the transport through the skin to molecules with certain properties such as low molecular weight (<500 Dalton), moderate lipophilicity (octanol-water partition coefficient between 10 and 1000), and modest melting point (<200° C.) correlating with good solubility. Even when an active substance exhibits such properties, it is usually necessary to find additional means to increase its transport across the skin.

Physical Enhancement Techniques

Hydration of the horny layer and addition of chemical enhancers that temporarily alter the barrier properties can enhance the flux of active substances. However, all these principles have clear limitations concerning the delivery of sufficiently high amounts of ionic molecules, large molecular weight, and substances with low potency. These limitations of chemical enhancement can be overcome to some extent by physical enhancement technologies.

Iontophoresis

Iontophoresis is an active process which has been extensively studied for years. It was first described by LeDuc in 1908, and has since found commercial use in the delivery of ionically charged compounds such as pilocarpine, dexamethasone, and lidocaine. In this delivery method, ions bearing a positive charge are driven across the skin at the site of an electrolytic electrical system positive electrode, while ions bearing a negative charge are driven across the skin at the site of an electrolytic electrical negative electrode.

The basic principle of iontophoresis is that a small electric current is applied to the skin. This provides the driving force to primarily enable penetration of charged molecules into the skin. A drug reservoir is placed on the skin under the active electrode with the same charge as the penetrant. Another electrode is positioned elsewhere nearby on the body either to function as circuit completion or it could be used to iontophoretically transport opposite charged therapeutic substances at the same time. The active electrode effectively repels the active substance and forces it into the skin. This simple electrorepulsion is known as the main mechanism responsible for penetration enhancement by iontophoresis. The number of charged molecules which are moved across the barrier correlates directly to the applied current and thus can be controlled by the current density.

Other factors include the possibility to increase the permeability of the skin barrier in the presence of a flow of electric current and electroosmosis. Contrary to electrorepulsion, electroosmosis can be used to transport uncharged and larger molecules.

Electroosmosis results when an electric field is applied to a charged membrane such as the skin and causes a solvent flow across this membrane. This stream of solvent carries along with it dissolved molecules. It enhances the penetration of neutral and especially polar substances.

Microneedles/Microcuts

In recent years, several attempts have been made to enhance the transport of substances across the skin barrier passively using minimally invasive techniques. The proper function of an appropriate system requires that the thickness of the stratum corneum (10 to 20 μm) has to be breached. More recent developments focus on the concept of microneedles/microcuts. Microneedles are needles that are 10 to 200 μm in height and 10 to 50 μm in width. They could be solid or hollow and are connected to a reservoir which contains the delivery substance.

Microneedle arrays are applied to the skin surface so that they pierce the upper epidermis deep enough to increase skin permeability and allow drug delivery, but too shallow to cause any pain to the pain receptors in the dermis. Therefore there is no limitation concerning polarity and molecular weight of the delivered drug molecules.

Chemical Enhancers

Permeation enhancers are able to passively promote the transport of substances across the skin barrier by a variety of mechanisms. The most important are:
Extraction of lipids from the stratum corneum
Alteration of the vehicle/skin partitioning coefficient
Disruption of the lipid bilayer structure
Displacement of bound water
Loosening of horny cells
Delamination of stratum corneum

Chemical enhancers can be categorized into different groups. Solvents like alcohols, alkylmethyl sulfoxides, and polyols mainly increase solubility and improve partitioning coefficient. Moreover, some solvents, e.g. Dimethlysulphat (DMSO), ethanol, may extract lipids, making the stratum corneum more permeable. Oleic acid, Azone® (epsilon-Laurocapram), and isopropyl myristate are typical examples of chemical enhancers which intercalate into the structured lipids of the horny layer where they disrupt the packing. This effect makes the regular structure more fluid and thus increases the diffusion coefficient of the permeant. Ionic surfactants, decylmethyl sulfoxide, DMSO, urea interact with the keratin structure in the corneocytes. This opens up the tight protein structure and leads to an increased diffusion coefficient mainly for those substances which use the transcellular route.

Fundamental Basics of a Galvanic Cell/Voltaic Cell

simple galvanic cell/Voltaic cell (Hereafter, interchangeable since they both refer to the same principle) is comprised of electrodes (two plates or rods, each made from a different kind of metal or metallic compound) are placed in an electrolyte solution. Electrolytes can be divided into acids (H₂CO₃, HCl, H₂SO₄, HNO₃, CH₃COOH . . . ), bases (NH₄OH, KOH, NaOH . . . ), and salts (NaCl, CaCl₂, NH₄Cl, KBr, CuSO₄, NaCH₃COO (sodium acetate), CaCO₃, NaHCO₃ (baking soda) . . . ) because they all give ions when dissolved in water.

Electrolytes can either be organic or inorganic substances or compounds. The metal in the anode (the negative terminal) oxidizes releasing negatively charged electrons and positively charged metal ions. If the two electrodes are externally connected, electrons will travel through the external wire (and the electrical load) to the cathode (the positive terminal). The electrons then combine with the material in the cathode. This combination process is called reduction, and it releases a negatively charged metal-oxide ion. At the interface with the electrolyte, this ion causes a water molecule to split into a hydrogen ion and a hydroxide ion. The positively charged hydrogen ion combines with the negatively charged metal-oxide ion and becomes inert. The negatively charged hydroxide ion flows through the electrolyte to the anode where it combines with the positively charged metal ion, forming a water molecule and a metal-oxide molecule.

In effect, metal ions from the anode will “dissolve” into the electrolyte solution while hydrogen molecules from the electrolyte are deposited onto the cathode.

When the anode is fully oxidized or the cathode is fully reduced, the chemical reaction will stop and the battery is considered to be discharged.

The amount of voltage and current that a galvanic cell produces is directly related to the types of materials used in the electrodes and electrolyte. The length of time the cell can produce that voltage and current is related to the amount of active material in the cell and the cell's design.

Every metal or metal compound has an electromotive force, which is the propensity of the metal to gain or lose electrons in relation to another material. Compounds with a positive electromotive force will make good anodes (Magnesium, Aluminium, Duralumin, Zinc, Cadmium, Iron, Chromium iron alloys, Chromium nickel iron alloys, Tin) and those with a negative force will make good cathodes (Platinum, Gold, Silver, Copper, Nickel copper alloys, Bronze, Brass, Nickel, Lead). The larger the difference between the electromotive forces of the anode and cathode; the greater the amount of electrical energy/current that can be produced by the cell.

The electrical power source of the present invention utilizes the principle of Galvanic cell at a micro level to provide electricity for the iontophoretic process.

Existing patented lontophoresis devices conventionally include two electrodes attached to a patient. Medication is placed under one or both of the electrodes, for delivery into the body as the instrument is activated. The power sources for these instruments are usually provided by external DC power supplies or batteries. These existing iontophoresis devices are disadvantaged by the fact that they are very bulky, which limits patient mobility and ability to conduct normal daily activity. The advantage of the present invention dermal patch is that it is a conveniently disposable, small skin patch. It effectively facilitates iontophoretic absorption through skin by utilizing thousands of micro-electrodes instead of two big electrodes. Most importantly, it is self-contained system including its own electrical energy supply. In addition, a dermal patch of the present invention not only utilizes iontophoresis but also other well-studied skin permeation enhancement techniques in order to maximize its effectiveness in delivering therapeutic agents across the skin barrier.

### SUMMARY OF THE INVENTION

The present invention generally concerns transdermal delivery of drugs by using a self-contained, disposable skin patch. The disposable percutaneous absorption patch of present invention is a small skin worn patch that contains drugs/therapeutic agents, permeation enhancers, and a self-powered iontophoretic delivering system which including thousands of micro-needle electrodes as well as a built in DC power supply, the Voltaic cell system. In one perspective, the present invention utilizes the principles of micro-current iontophoresis, epidermal micro-needles, and permeation enhancers as means of introducing drugs conveniently, painlessly, quickly, and effectively through the skin barrier. In another perspective, the present invention utilizes the principle of Voltaic cell at a micro level as a mean of producing its own electricity, which is a crucial element of the iontophoretic process.

### BRIEF DESCRIPTION OF THE DRAWINGS

FIG. 1: A perspective view of the dermal patch of the present invention

FIG. 2: A transverse cross section of the dermal patch of the present invention which shows components of the dermal patch

FIG. 3: An exploded view of the dermal patch's transverse cross section which shows components in greater details.

FIG. 4: A perspective view of the needle sheet of the present invention

FIG. 5: An exploded view cross section of a portion of the needle sheet which shows a micro-needle in details.

### DETAILED DESCRIPTION OF THE PRESENT INVENTION

It is an important aspect of the present invention that it provides a complete, self-contained transdermal drug delivery skin patch, which utilizes the principle of iontophoresis in a novel way. It is a small, bandage-like, pre-packaged dermal patch FIG. I that includes all of the components FIG. 2 necessary for iontophoretic delivery of therapeutic agents across the skin barrier conveniently, painlessly, quickly, and effectively.

The key element of the present invention is the micro-needles 10 (10A and 10B); Hereafter, a micro-needle also referred as micro-electrode interchangeably since it basically functions as an electrode. They simultaneously act as micro-electrodes for the micro-current Galvanic cell system and as electrolytic micro-electrodes for the iontophoretic system of the present invention dermal patch. In addition, these micro-needles 10 also function as micro-scalpels which making multiples of micro-cuts/holes through the stratum corneum to enhance absorptions, passive absorption in particular.

The Voltaic cell system of the present invention comprised of arrays of micro-electrodes 10 and electrolytic solution layer 5. The Voltaic cell system supplies DC (Hereafter referred as Direct Current) micro-current for the iontophoretic system.

The iontophoretic system of the present invention comprised of arrays of micro-electrodes 10, a DC power supply, and a medication layer 8 which contains the therapeutic agents of choice to be iontophoretically transported. The DC power supply is the Voltaic cell system mentioned above.

The micro-needle 10 has a special shaped structure including the head 11, body 12, and pointed tip 13 portions FIG. 5. The head portion of a micro-needle 11 is a flatten-spherical structure. This flatten-spherical shaped structure optimally maximizes the contact surface area of the needle head with the electrolytic substance 5. Since the larger the contact surface area of an electrode 10 with the electrolyte 5; the greater the electrochemical reaction with the electrolyte. The greater the reaction the greater the amount of electrical charges/currents being produced at each electrode 10. Also the spherical shaped head 11 of the micro-needle 10 has another crucial advantage in that it allows electrical charges being highly concentrated at the needle's tip 13. Since the higher the electrical charges/currents concentrated at the needle tip 13 the stronger the electrorepulsive force and therefore the amount of substance can be iontophoretically delivered across the skin barrier during a period of time.

There are two groups of micro-needles 10; one group of needles 10A is made of one type of metal/compound with a positive electromotive force (Magnesium, Aluminium, Duralumin, Zinc, Cadmium, Iron, Chromium iron alloys, Chromium nickel iron alloys, Tin . . . ) which act simultaneously as anodes 10A for the Voltaic cell component and negative electrode 10A for the iontophoretic system of the present invention's dermal patch; the other group of needles 10B are made of a different type of metal/compound with a negative electromotive force (Platinum, Gold, Silver, Copper, Nickel copper alloys, Bronze, Brass, Nickel, Lead) which act simultaneously as cathodes 10B for the Voltaic cell component and as a positive electrode 10B for the iontophoretic system of the present invention's dermal patch;

The greater the difference of the electromotive force between an anode's 10A and a cathode's 10B material the greater the electrical potential would be created between the two electrodes. Therefore, electrode material is one of the important factors that can be selectively chosen to partly determine the rate of iontophoretic transport.

The micro-needles 10 are embedded in a thin sheet 7 of rectangular shaped, or other configurations, made of soft, flexible, hypoallergic natural or polymer plastic/vinyl; the sheet has said top and bottom surfaces; there are rows of micro-needles 10 embedded into and cover the whole surface of the needle sheet. In each row, the micro-needles 10 are being specially arranged in that an anode micro-needle 10A is alternately position between two cathode micro-needles 10B and vice versa FIG. 4. The number and type of micro-needles 10 per row, the ratio of two types of micro-needles 10A:10B, and the distance between rows can be varied and modified depending on many factors such as the electrical charge characteristics of the therapeutic agents, targeted rate of absorption . . . etc. The spherical shaped head 11 of each micro-needle is protruded and exposed on the top surface of the needle sheet where it comes to contact the electrolytic solution 5 during application. The needle body 12 is tightly embedded inside the needle sheet 7 so that the electrolytic solution cannot leak through. The needle's tip 13 is protruded and exposed on the bottom side of the needle sheet 7 where it comes in contact with the skin surface and with the therapeutic agents during application.

The electrolytic medium 8 of the Voltaic cell component of the present invention could be an aqueous solution or a gelatinous substrate contains a dissolved electrolyte. The electrolyte could be one of the following: acidic electrolytes (H₂CO₃, HCl, H₂SO₄, HNO₃, CH₃COOH . . . ), basic electrolytes (NH₄OH, KOH, NaOH . . . ), and salt electrolytes (NaCl, CaCl₂, NH₄Cl, KBr, CuSO₄, NaCH₃COO (sodium acetate), CaCO₃, NaHCO₃ (baking soda) . . . ). At one level, the degree of fluidity or viscosity of the electrolytic solution/gel partly determines the rate of the electrochemical reaction between the electrolytic ions and electrodes. The lower the fluidity or viscosity level of electrolytic medium the faster the reaction between the electrolytic ions and electrodes since the electrolytic ions are moving around easier in a low viscous solution/gel. The faster the reaction the greater the amount of charge/electrical current that are being produced at an electrode during a period of time. A greater charged electrode, in turn, possesses a higher iontophoretic transport capability. A similar but opposite analogy is true for a higher viscosity electrolytic solution/gel. Therefore, the degree of fluidity or viscosity is one of the controlled factors that could be selectively manipulated to enhance the iontophoretic absorption rate. At another level, the electrolyte can also selectively chosen to controllably determine the rate of iontophoretic transport since each electrolyte has a different degree of ionization.

The aqueous electrolytic solution/gel 5 is enclosed within a thin, leakage-proof, rupturable membrane 6 of natural or synthetic polymer. The cover membrane 6 prevents premature interaction between the needle electrodes 10 and the electrolytic solution/gel 5 before usage/application. The enclosed electrolytic solution/gel 5 is positioned on top of the needle sheet 7.

The medication layer 8 which is a thin gelatinous layer located on the bottom surface of the needle sheet 7; it is comprised of mixtures of therapeutic agents and permeation enhancers of choice dissolved in a hydrophilic gel substrate; The gel substrate of the medication layer is relatively rigid so it would not drop off when in an upside down position during the process of application to patient's skin yet the gel substrate is easily being emulsified by a squeezing action into an amorphous, more fluidity form during application. The emulsified state of medication gel would facilitate absorption both actively and passively since molecules of therapeutic agents and other active agents would be able to move around much easier together with increasing contact surface area with the skin by filling microcuts/holes and natural skin folds/cleavages.

The gelatinous substrate for the medication layer 8 and electrolytic gel 5 could be a hydrogel or a substance selected from the group consisting of agarose, polyvinyl alcohol, polyvinylpyrrolidone, methyl cellulose, hydroxypropyl methylcellulose and, carboxymethyl cellulose, and combinations thereof;

The electrolyte layer 5, the needle-sheet 7, and the medication layer 8 are stacked on top of each other from top to bottom respectively FIG. 2 and 3.

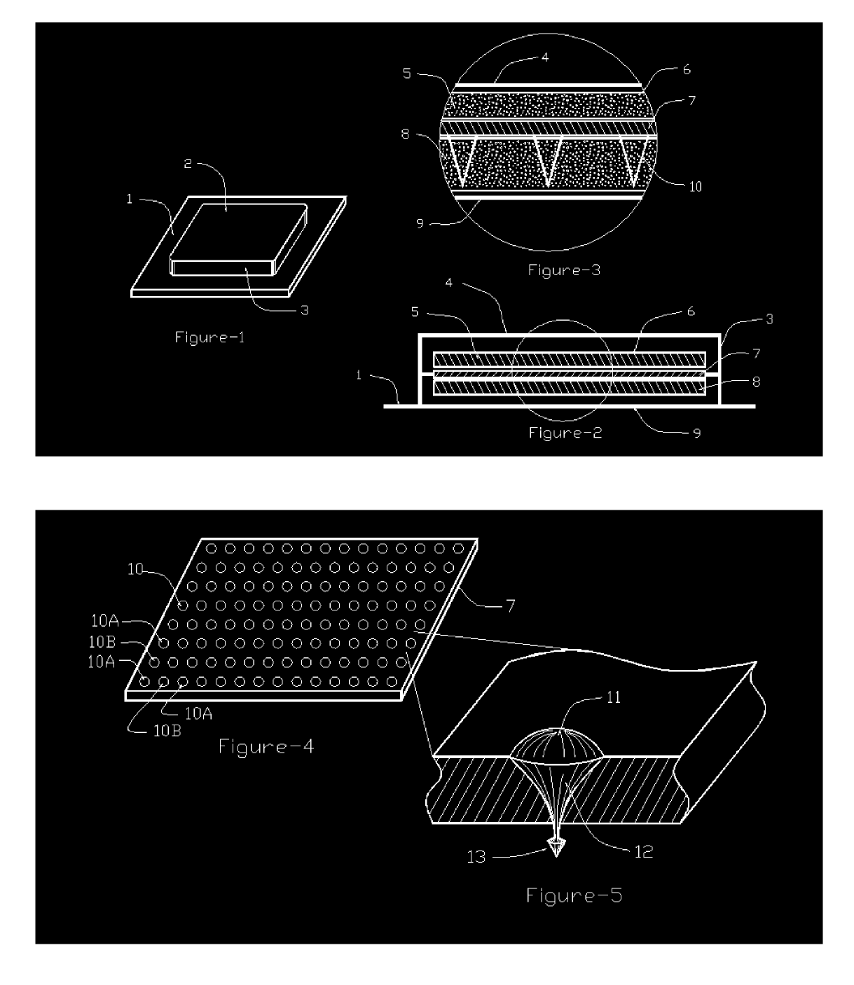

The outer structure of the present invention dermal patch FIG. 1 is a self-supporting structure to give the patch the required integrity and strength by acting as a supporting framework for other components. The outer structure framework includes a built in chamber 2 and a skin adhesion peripheral area 1; the chamber covers all the layers of gel 5, 6 and needle sheet 7; the chamber 2 portion is made of a soft, flexible, thin sheet of hypoallergic natural or synthetic polymer which could be transparent, opaque or colors; The chamber 2 is opened on the bottom surface where the medication gel layer 8 would come into contact with patient's skin; The skin adhesion portion 1 is a soft, flexible, porous fabric or polymer with a thin layer of hypoallergic adhesive on the skin contact surface, which adhesively holds the dermal patch onto skin surface during application.

The peripheral edges of needle sheet 7 are tightly glued to the walls 3 of chamber to prevent mixing between electrolyte gel and medication gel during application FIG. 2.

A cover sheet 9 is a thin plastic sheet that covers the exposed content, which is the medication gel layer 8, of the chamber 2 and the skin adhesive portion of the dermal patch on the bottom/skin contact surface; this cover sheet 9 is peeled off when the patch is readily being applied to skin; the cover sheet's 9 function is to protect the chamber's 2 contents and the skin adhesive portion 1.

The whole structure of the dermal patch FIG. I might be enclosed or stored in hard plastic case which provides protection from crushing of the gel layers.

When application is desired, the skin area where the patch will be placed is cleaned with alcohol or other antiseptic solutions. The patch is peel off from its plastic cover 9 and placed on top of the prepared skin area. The patient himself/herself or a healthcare provider uses his or her finger tips to slightly press the peripheral adhesive portion 1 down onto the skin. When the adhesive 1 portion is tightly sealed onto the skin surface, using finger tips to lightly press and rub in a circular motion on the top surface 4 FIG. 2 of the chamber. This pressing and rubbing actions will rupture the covered membrane 6 of the electrolytic solution/gel layer 5 and help to emulsify the rigid medication gel layer 8 below. Also this pressing action will push the tips of micro-needles 13, which are about 10 to 200 μm in height and 10 to 50 μm in width, through the stratum corneum layer of the skin creating hundreds of micro-cuts/micro-holes where the emulsified medication gel 8 would fill in and being transported both actively and passively across the skin.

As soon as the electrolytic solution/gel's 5 covered membrane 6 is ruptured, the electrolytic solution/gel is spreading all over the top surface of the needle sheet 7 where the heads of micro-needles 11 are now being submerged within the electrolytic solution/gel 5. At that instant, the electrochemical interaction between electrolytic ions and micro-needle electrodes 10 begin to create an electrical potential between anodes 10A and cathodes 10B micro-needle electrodes. In another way, the anode electrodes 10A become negatively charged and the cathode electrodes 10B become positively charged. At the same moment, the needle tips 13 of anode electrodes 10A begin iontophoretically pushing negatively charged molecules of therapeutic agents across the skin barrier. Similarly, the needle tips 13 of cathode electrodes begin iontophoretically pushing positively charged molecules of therapeutic agents across the skin barrier.

In addition, the micro-cuts/holes effectively alter the integrity of the stratum corneum, therefore, further facilitate the passive absorption of both charged and non-charged molecules of the therapeutic agents across the skin barrier.

If permeation enhancers are included in the medication gel 8, the passive absorption of both charged and non-charged molecules of therapeutic agents would be further facilitated.

During the time of application, patient is advised to occasionally rub in circular motions on the top surface 4 of the chamber 2 in order to enhance mixing of electrolytic substance, therapeutic agents and others thus further effectively facilitating both active and passive absorption processes. Since occasional mixing of electrolytic solution would enhance interaction of fresh electrolytic molecules and micro-electrodes of Voltaic cell system; therefore, increasing electrical production which in turn boosting the iontophoretic process. In a similar manner, occasional mixing of the medication gel layer 8 would enhance interaction of therapeutic agents with micro-needles' tips 13; therefore, increasing the effectiveness of iontophoretic transport.

In addition, rubbing would also enhance passive absorption processes as well; since more fresh active molecules are coming into contact with the skin. Also as the blood circulation of the skin is well circulated, the passive absorption process would in turn be greatly enhanced since the absorbed therapeutic agents are quickly transported away the skin area thus favorably increasing the concentration gradient of the therapeutic agents across the skin barrier.

The foregoing is considered as illustrative only of the principles of the invention. Further, since numerous modifications and changes will readily occur to those skilled in the art, for instance, the size and shape of the micro-needles, the needles' tips in particular, the number and ratio of different types of micro-needles, the location and arrangement of micro-needles on the needle sheet, the materials of choice . . . etc. It is not desired to limit the invention to the exact construction and operation shown and described, and accordingly all suitable modifications and equivalents may be resorted to, falling within the scope of the invention.

What is claimed is:

1. A disposable, self-contained percutaneous absorptive patch comprised of the following built in systems and components:

1. Said a Voltaic cell system of the present invention comprised of said micro-electrodes and said electrolytic solution; The Voltaic cell system supplies DC current for said the iontophoretic system;

2. Said iontophoretic system of the present invention comprised of said micro-electrodes, a DC (Hereafter referred as Direct Current) power supply, and said medication layer which contains substances to be iontophoretically delivered, hereafter referred as therapeutic agents of choice; The DC power supply is the Voltaic cell system claimed 1 above;

3. Specially-designed shaped, said micro-needles which essentially serves as three crucial functions: acting as electrodes for Voltaic system in claim 1 and as electrolytic electrodes for iontophoretic system in claim 2; In addition, they also function as said microscalpels;

4. There are said two groups of micro-needles; one group of needles are made of one type of metal/compound with a positive electromotive force (Magnesium, Aluminium, Duralumin, Zinc, Cadmium, Iron, Chromium iron alloys, Chromium nickel iron alloys, Tin) which act simultaneously as said anodes, hereafter, referred as the anodes for the Voltaic cell system as well as negative electrolytic electrodes for the iontophoretic system of the present invention's dermal patch; the other group of needles are made of a different type of metal/compound with a negative electromotive force (Platinum, Gold, Silver, Copper, Nickel copper alloys, Bronze, Brass, Nickel, Lead) which act simultaneously as said cathodes, hereafter, referred as cathodes for said the Voltaic cell system as well as positive electrolytic electrodes for said the iontophoretic system of the present invention's dermal patch;

5. The micro-needles have a specially designed shape including a big said flatten-spherical shaped head portion, said body portion, and said a pointed tip; The special shape of micro-needles serves as two crucial functions: intentionally allowing the concentration of electrical charges to be highest at the tip of the needles and optimally maximizing its electricity production capability by optimally increasing the contact surface area with the electrolyte substance at the head portion of the needle;

6. Said a needle-sheet: a thin, rectangular shaped, or other configurations, sheet of soft, flexible, hypoallergic natural or polymer plastic/vinyl; the sheet has said top and bottom surfaces; there are said rows of micro-needles tightly embedded into the sheet;

7. Said an electrolytic solution/gel component constituted of an electrolyte dissolved in either an aqueous medium or a gelatinous substrate; The electrolyte could be one of the following organic or inorganic substances/compounds: acidic electrolytes (H₂CO₃, HCl, H₂SO₄, HNO₃, CH₃COOH . . . ), basic electrolytes (NH₄OH, KOH, NaOH . . . ), and salt electrolytes (NaCl, CaCl₂, NH₄Cl, KBr, CuSO₄, NaCH₃COO (sodium acetate), CaCO₃, NaHCO₃ (baking soda) . . . ); the electrolytic component is enclosed within a thin leakage-proof, rupturable membrane made of natural or synthetic polymer; The membrane prevents premature interaction between needle electrodes and electrolyte before usage; The electrolytic solution/gel is located on the top surface of the needle sheet;

8. Said medication layer which is a thin gelatinous layer located on the bottom surface of the needle sheet; it is comprised of mixtures of said therapeutic agents and said permeation enhancers of choice dissolved in a hydrophilic gel substrate; The gel substrate of said medication layer is relatively rigid so it would not drop off when in an upside down position during the process of application to patient's skin;

9. Said therapeutic agents are any pharmaceutical compounds/substances of choice, which are intentionally to be delivered into a patient's body;

10. Said permeation enhancers are any biochemical or chemical substances/compounds, which are able to safely and passively promote the transport of substances across the skin barrier;

11. Said the electrolyte layer, the needle-sheet, and the medication gel layer are stacked on top of each other from top to bottom respectively;

12. Said the outer structure framework includes a built in said chamber and a said skin adhesion peripheral area; the chamber covers all the layers of gel and needle sheet; the chamber portion is made of a soft, flexible, thin sheet of hypoallergic natural or synthetic polymer, which could be transparent, opaque or colors; The chamber is opened on the bottom surface; The skin adhesion portion is a soft, flexible, porous fabrics or polymer with a thin layer of hypoallergic adhesive on the skin contact surface;

13. The peripheral edges of said needle sheet are tightly glued to said the walls of the chamber to prevent mixing between electrolyte gel and medication gel during application

14. A said cover sheet is a thin plastic sheet that covers the exposed content, which is the medication gel layer, of the chamber and the skin adhesive portion of the dermal patch on the bottom/skin contact surface; this cover sheet is peeled off when the patch is readily being applied to skin; the cover sheet's function is to protect the chamber's contents and the skin adhesive portion;

15. Gelatinous substrate for both said the electrolytic layer and said the medication layer is either hydrogel or material selected from the group consisting of agarose, polyvinyl alcohol, polyvinylpyrrolidone, methyl cellulose, hydroxypropyl methylcellulose and, carboxymethyl cellulose, and combinations thereof.
